(19) United States
(12) Patent Application Publication
(10) Pub. No.: US 2024/0365802 A1
(43) Pub. Date: Nov. 7, 2024

(54) PROTEIN ENCAPSULATION OF NUTRITIONAL AND PHARMACEUTICAL COMPOSITIONS

(71) Applicant: Clover Corporation Limited, Altona North, Victoria (AU)

(72) Inventors: Glenn ELLIOTT, Casuarina (AU); Jessica RYAN, Coorparoo (AU); Lourdes URBAN-ALANDETE, Camp Hill (AU); Bo WANG, Parklea (AU); YunYun XU, Acacia Ridge (AU)

(21) Appl. No.: 17/754,842

(22) PCT Filed: Oct. 16, 2020
(86) PCT No.: PCT/AU2020/051120

§ 371 Date: Apr. 13, 2022

(30) Foreign Application Priority Data
Oct. 16, 2019 (AU) 2019903901

(51) Int. Cl. A23C 1/16; A23C 21/08

(52) U.S. Cl. CPC A23C 1/16; A23C 21/08

(57) ABSTRACT

The present disclosure relates to microencapsulated compositions comprising one or more hydrophobic materials encapsulated by an encapsulant, wherein the encapsulant comprises one or more modified proteins and/or peptides; and wherein the one or more modified proteins and/or peptides are obtained from a starting protein by subjecting the starting protein to a high shear process, such that the average particle size of the one or more modified proteins and/or peptides is reduced relative to the starting protein. The present disclosure further relates to methods for protecting a hydrophobic material from oxidative degradation, for improving the oxidative stability of a hydrophobic material, for reducing the surface free fat of a microencapsulated composition, and to stable emulsions and composition comprising a hydrophobic material

### TECHNICAL FIELD

The present disclosure broadly relates to encapsulated compositions suitable for both nutritional and pharmaceutical applications and to means for protecting hydrophobic materials in encapsulated compositions from oxidation and oxidative degradation.

### BACKGROUND OF THE DISCLOSURE

It is well known that various hydrophobic bioactive compounds such as long-chain polyunsaturated fatty acids (“LCPUFAs”), carotenoids, water-insoluble vitamins, phenolic compounds, flavours and aroma components provide various health benefits. In particular, LCPUFAs are an important nutritional component of the human diet and many people fail to consume an adequate amount of these essential fatty acids, in particular omega-3 fatty acids such as eicosapentaenoic acid (EPA) and docosahexaenoic acid (DHA). A large number of studies have found that omega-3 fatty acids play an influential role in heart, brain and eye health, and their dietary intake has been well associated with improved cardiovascular function and a reduction in various inflammatory-related conditions. For example, a recent study suggests that EPA and DHA may have the ability to decrease heart rate and oxygen consumption during exercise, therefore contributing to enhanced physical and mental performance in athletes (People et al., Journal of Cardiovascular Pharmacology, 2008, 52: 540-547). Because of their essential nutritional role, compositions comprising omega-3 fatty acids are important in terms of both nutritional supplementation, and as pharmaceutical agents.

Accordingly, there is a growing trend towards incorporating omega-3 fatty acids, for example, fish oils, algal oils and some plant seeds oils, into food products to promote public health. However, due to the susceptibility of these fatty acids to oxidation or degradation upon exposure to oxygen, elevated temperature or light, which are common occurrences during food production and storage, it is a challenge to successfully fortify products with omega-3 fatty acids, maintaining stability and activity of the omega-3 fatty acids. The oxidation and/or degradation of omega-3 fatty acids generates undesirable oxidation breakdown products which may adversely affect the organoleptic properties or physiological properties of the formulation. As such, it is challenging to produce, transport and store these functional foods.

Microencapsulation technology, through which bioactive compounds can be entrapped within physical protective shell materials, has been successfully used to protect omega-3 fatty acids against oxidation and degradation. Spray drying is the most widely used technique to produce microcapsule powders. Typically, spray dried microcapsule powders containing omega-3 rich oils and have an oil loading of approximately 30% (w/w) and a surface free fat content of approximately 1% (w/w). Due to the superior functional properties of Maillard reaction products (MRPs), omega-3 oil-containing microcapsule powders have been produced with an oil loading as high as 48±2%, while maintaining a surface free fat content of approximately 1% (w/w): however, such products exhibit an induction period (the number of hours before onset of oxidation of encapsulated oil) of typically just 50 hours.

There is a need for the development of encapsulation and delivery systems capable of improving the oxidative stability of hydrophobic compounds, and especially omega-3 oils.

### SUMMARY OF THE DISCLOSURE

The present disclosure is predicated on the inventors' unexpected discovery that use of an encapsulant comprising one or more modified proteins and/or peptides, wherein the modified protein(s) and/or peptide(s) is obtained from a starting protein by subjecting the starting protein to a high shear process, can provide compositions comprising hydrophobic materials having both a particularly high oxidative stability and an especially low surface free fat content (i.e. a high oil encapsulation efficiency). In particular embodiments, the one or more modified proteins and/or peptides is obtained from a starting protein by subjecting the starting protein to a high shear process such that the average particle size of the modified protein(s) and/or peptide(s) is reduced relative to the starting protein. In particular embodiments, the average particle size of the modified protein(s) and/or peptide(s) is about 70% of the average particle size of the starting protein or less, for example about 65% of the average particle size of the starting protein or less. In some embodiments, one or more modified proteins and/or peptides are used in a composition or method of the present disclosure, wherein the one or more modified proteins are obtained from one or more respective starting proteins.

A first aspect of the present disclosure provides a microencapsulated composition comprising one or more hydrophobic materials, wherein the encapsulant comprises one or more modified proteins and/or peptides, and wherein the modified protein(s) and/or peptide(s) is obtained from a starting protein by subjecting the starting protein to a high shear process, such that the average particle size of the modified protein(s) and/or peptide(s) is reduced relative to the starting protein. In preferred embodiments, the average particle size of the modified protein(s) and/or peptide(s) is about 70% of the average particle size of the starting protein or less, for example about 65% of the average particle size of the starting protein or less.

According to some preferred embodiments, the microencapsulated composition has a surface free fat content of less than about 1.8%, for example less than about 1%, for example less than about 0.8%.

According to some embodiments, the high shear process is carried out at alkaline pH, for example a pH of about 8. In some embodiments, the high shear process comprises subjecting the starting protein to a pressure of from about 20 mPa to about 300 mPa. In some embodiments, the high shear process is a homogenisation process. In some embodiments, the high shear process is a microfluidisation process.

According to some embodiments, the one or more modified proteins and/or peptides are in the form of a protein fraction. According to some embodiments, the modified protein is modified whey protein.

According to some embodiments, the encapsulant further comprises one or more carbohydrates, for example glucose syrup and dextrose monohydrate, or a combination thereof.

According to some embodiments, the one or more modified proteins and/or peptides are present at from about 3% w/w to about 25% w/w based on the total weight of the composition.

According to some embodiments, the ratio of the modified protein component of the encapsulant to the carbohydrate component of the encapsulant is in the range of about 1:10 to 1:1.

According to some embodiments, the hydrophobic material is an edible oil. In some embodiments, the hydrophobic material comprises one or more long-chain polyunsaturated fatty acids (LCPUFAs). In some embodiments, the LCPUFAs comprise omega-3 fatty acids and/or omega-6 fatty acids. In some embodiments, the LCPUFAs are present in triglyceride form. In some embodiments, the LCPUFAs are present in one or more LCPUFA-containing oils; in some embodiments, the one or more oils comprise a fish oil. In some such embodiments, the fish oil is tuna oil.

According to some embodiments, the composition further comprises at least one source of vitamin C.

In some embodiments, the composition is in the form of an oil-in-water emulsion. In some embodiments, the composition is in the form of a spray-dried powder.

According to a second aspect, the present disclosure provides a method for protecting a hydrophobic material from oxidative degradation, comprising:
subjecting a starting protein to a high shear process producing one or more modified proteins and/or peptides, such that the average particle size of the one or more modified proteins and/or peptides is reduced relative to the starting protein; and
encapsulating the one or more hydrophobic materials with an encapsulant comprising the one or more modified proteins and/or peptides.

According to a third aspect, the present disclosure provides a method for improving the oxidative stability of a hydrophobic material, comprising:
subjecting a starting protein to a high shear process producing one or more modified proteins and/or peptides, such that the average particle size of the one or more modified proteins and/or peptides is reduced relative to the starting protein; and
encapsulating the one or more hydrophobic materials with an encapsulant comprising the one or more modified proteins and/or peptides.

According to a fourth aspect, the present disclosure provides a method for reducing the surface free fat of a microencapsulated composition comprising one or more hydrophobic materials encapsulated by an encapsulant, comprising subjecting a one or more starting proteins and/or peptides to a high shear process producing one or more modified proteins and/or peptides, such that the average particle size of the one or more modified proteins and/or peptides is reduced relative to the one or more starting proteins and/or peptides; and encapsulating the one or more hydrophobic materials with an encapsulant comprising the one or more modified proteins and/or peptides.

In some embodiments of methods according to the second, third or fourth aspects, the average particle size of the one or more modified proteins and/or peptides is about 70% or less of the average particle size of the starting protein, for example about 65% or less.

In some embodiments, the high shear process is carried out at alkaline pH, for example at a pH of about 8.

In some embodiments, the hydrophobic material comprises one or more LCPUFAs, for example in triglyceride form.

According to a fifth aspect, the present disclosure provides a stable emulsion comprising a hydrophobic material, wherein said emulsion further comprises one or more modified proteins and/or peptides, and wherein the one or more modified proteins and/or peptides are obtained from a starting protein by subjecting the starting protein to a high shear process, such that the average particle size of the one or more modified proteins and/or peptides is reduced relative to the starting protein.

In some embodiments, the average particle size of the one or more modified proteins and/or peptides is about 70% or less of the average particle size of the starting protein, for example about 65% or less.

In some embodiments, the high shear process is carried out at alkaline pH, for example at a pH of about 8.

In some embodiments, the hydrophobic material comprises one or more LCPUFAs, for example in triglyceride form.

According to a sixth aspect, the present disclosure provides a composition comprising a hydrophobic material and one or more modified proteins and/or peptides, wherein the one or more modified proteins and/or peptides are obtained from a starting protein by subjecting the starting protein to a high shear process, such that the average particle size of the one or more modified proteins and/or peptides is reduced relative to the starting protein.

### BRIEF DESCRIPTION OF THE FIGURES

Exemplary embodiments of the present disclosure are described herein, by way of non-limiting example only, with reference to the following drawings.

FIG. 1. Interfacial tension of corn oil with 1.0% w/w uWPI and mWPI (native and pH 8 solutions).

FIG. 2. Scheme showing the process for preparing unmodified whey protein isolate-based microencapsulated powder (uWPI powder) and modified whey protein isolate-based microencapsulated powder (mWPI powder).

FIG. 3. Graph showing Oxipres analysis results of omega-3 oil-containing microencapsulated uWPI and mWPI powders compared to omega-3 oil-containing microcapsule powders encapsulated with Maillard reaction products (MRPs).

FIG. 4. Overall quality of microencapsulated uWPI and mWPI powders over a 4-week rapid exposure period.

FIG. 5. Rancid and Marine odours and flavours of microencapsulated uWPI & mWPI powders over a 4-weeks rapid exposure period.

### DETAILED DESCRIPTION

Throughout this specification, unless the context requires otherwise, the word “comprise”, or variations such as “comprises” or “comprising ”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” is understood to refer to a range of numbers that a person of skill in the art would consider equivalent to the recited value in the context of achieving the same function or result.

In the context of this specification, the terms “a” and “an” refer to one or to more than one (i.e. to at least one) of the grammatical object of the article. By way of example, “an element” means one element or more than one element.

The term “protein” means a polymer made up of amino acids linked together by peptide bonds. The term “peptide” may also be used to refer to such a polymer although in some instances a peptide may be shorter (i.e. composed of fewer amino acid residues) than a protein. The terms “protein” and “peptide” may be used interchangeably herein.

As used herein, the term “oxidative stability” in relation to hydrophobic materials and compounds, for example LCPUFAs, means the stability of the hydrophobic material, for example LCPUFAs or a LCPUFA-containing oil, in the presence of oxygen and resistance to oxidation or oxidative degradation. Thus, a higher oxidative stability is indicative of greater resistance to oxidation and oxidative degradation. Typically, reference to improved oxidative stability resulting from encapsulation in accordance with the present disclosure means an improvement over the oxidative stability observed in the absence of an encapsulant according to the present disclosure or in the presence of an alternative encapsulant.

In accordance with the embodiments of the present disclosure, modified whey protein was used as an encapsulant in a microencapsulated composition comprising tuna oil. Modification of whey protein isolate by a high shear process led to a reduction of its average particle size, thought to be due to the separation of water soluble protein aggregates. In particular, average particle size of the modified protein was about 51% of that of the starting protein, both at native pH and alkaline pH (pH 8).

This modified protein was used as a key encapsulant component to provide a spray dried microencapsulated powder to stabilise omega-3 oils at high levels of total oil loading (>45% total oil loading (w/w) (tuna oil), in particular about 49%), and low surface free fat content of less than 1.5±0.1% (1.3±0.5% and, at pH 8, an even lower surface fat free content of 0.6±0.1%). The resulting microencapsulated powders also exhibited very high oxidative stability, with an induction period of well over 100 hours, and acceptable primary and secondary oxidative properties (including peroxide value, p-anisidine value, overall quality and rancid and marine odours) over a 4 week rapid exposure period. The modification process disclosed herein may be applied to a range of proteins, to provide microencapsulation systems which can be used to extend the shelf life of various susceptible hydrophobic compounds including omega-3 oils, carotenoids, water-insoluble vitamins, phenolic compounds, flavours and aroma components.

Accordingly, particular embodiments of the present disclosure provide microencapsulated compositions comprising one or more hydrophobic materials, wherein the encapsulant comprises a modified protein and/or peptide, and wherein the modified protein and/or peptide is obtained from a starting protein by subjecting the starting protein to a high-shear process, such that the average particle size of the modified protein and/or peptide is reduced relative to the starting protein.

Also provided are methods and compositions in which a modified protein or peptide is used to encapsulate one or more hydrophobic materials, to protect the one or more hydrophobic materials from oxidation or oxidative degradation. The protection from oxidation or oxidative degradation may be determined by any suitable means well known to those skilled in the art.

Microencapsulated compositions of the present disclosure may be in the form of, for example, an emulsion or may be in a solid form. The emulsion may comprise an oil-in-water emulsion. The solid form may be a powder. The powder may be obtained by spray drying, for example of an emulsion. In one embodiment, the composition is a free-flowing powder. The powder may have a mean particle size between about 10 μm and 1000 μm, or between about 50 μm and 800 μm, or between about 100 μm and 300 μm. In alternative embodiments the composition may be in the form of granules.

Compositions of the present disclosure are generated by microencapsulation, wherein the encapsulant comprises or consists of a modified protein and/or peptide. A “modified protein” or “modified peptide” is obtained from a starting protein by subjecting the starting protein or peptide to a high shear process, such that the average particle size of the modified protein or peptide is reduced relative to the starting protein. In some embodiments, one or more modified proteins is used in a composition or method of the present disclosure, wherein the one or more modified proteins and/or peptides are obtained from one or more starting proteins respectively.

Reference within this specification to a “protein” may refer to a starting protein, a modified protein, or both, as will be readily understood by the relevant context.

A high shear process may be used to alter one or more properties of a protein, such as its particle size, solubility, foaming, gelling and/or emulsifying properties. Fat globule size of emulsions formed with aqueous solutions of such proteins and fats may also be reduced, and interfacial tension with oils may be reduced, in particular when the modified protein is subjected to a high shear process and used in aqueous solution at an alkaline pH, for example a pH of about 8. Any suitable high shear process may be used to modify the protein, and a variety of high shear processes will be familiar to a person skilled in the art.

In some embodiments, the high shear process is a homogenisation process. In some exemplary embodiments, the homogenisation process may be a high pressure homogenisation process wherein the protein is forced to flow at high velocity through a narrow gap. In some embodiments, the homogenisation process is microfluidisation. A microfluidisation process uses high shear rates and uniform processing pressures, and advantageously provides consistent nano-scale particle sizes and narrow particle size distributions. Exemplary microfluidisation apparatus are available from Microfluidics International Corporation, USA. In some exemplary embodiments, the homogenisation process may be an ultrasonic pressure homogenisation process, wherein sonic pressure waves are generated in a media to cause homogenisation. In some embodiments, the homogenisation process may be a mechanical homogenisation process, such as the use of a rotor-stator homogeniser, for example with multiple stages, or a blade-type homogeniser. The skilled addressee will appreciate that the scope of the present disclosure is not limited by reference to any specific homogenisation process.

In some embodiments, the high shear process comprises multiple passes through the high-shear arrangement. For example, in embodiments using a high pressure homogenisation process, a substance may undergo multiple passes through the narrow gap to achieve the desired homogenisation. For example, the high shear process may comprise 1, 2, 3, 4, 5, 6, 7, 8, 9, 10 or more passes.

In some embodiments, the high shear process comprises subjecting the starting protein to a pressure of from about 20 mPa to about 300 mPa, for example from about 50 mPa to about 300 mPa, for example from about 100 mPa to about 300 mPa, for example from about 100 mPa to about 250 mPa, for example from about 100 mPa to about 200 mPa, for example from about 125 mPa to about 175 mPa, for example about 150 mPa, or, alternatively, from about 125 mPa to about 300 mPa, for example from about 150 mPa to about 300 mPa, for example from about 175 mPa to about 300 mPa, for example from about 200 mPa to about 300 mPa, for example from about 225 mPa to about 300 mPa, for example from about 250 mPa to about 300 mPa.

In some particular embodiments, the high shear process comprises a homogenisation process, the homogenisation comprising subjecting the starting protein to a pressure of from about 20 mPa to about 300 mPa, for example from about 50 mPa to about 300 mPa, for example from about 100 mPa to about 300 mPa, for example from about 100 mPa to about 250 mPa, for example from about 100 mPa to about 200 mPa, for example from about 125 mPa to about 175 mPa, for example about 150 mPa, or, alternatively, from about 125 mPa to about 300 mPa, for example from about 150 mPa to about 300 mPa, for example from about 175 mPa to about 300 mPa, for example from about 200 mPa to about 300 mPa, for example from about 225 mPa to about 300 mPa, for example from about 250 mPa to about 300 mPa.

In some particular embodiments, the high shear process comprises a homogenisation process, the homogenisation comprising subjecting the starting protein to a pressure of from about 20 mPa to about 300 mPa, for example from about 50 mPa to about 300 mPa, for example from about 100 mPa to about 300 mPa, for example from about 100 mPa to about 250 mPa, for example from about 100 mPa to about 200 mPa, for example from about 125 mPa to about 175 mPa, for example about 150 mPa, or, alternatively, from about 125 mPa to about 300 mPa, for example from about 150 mPa to about 300 mPa, for example from about 175 mPa to about 300 mPa, for example from about 200 mPa to about 300 mPa, for example from about 225 mPa to about 300 mPa, for example from about 250 mPa to about 300 mPa, for 1, 2, 3, 4, 5, 6, 7, 8, 9, 10 or more passes.

In some particular embodiments, the high shear process is carried out in an alkaline environment, such as an alkaline aqueous solution, for example the protein is subjected to the high shear process in an aqueous solution at a pH of about 8.

Subjecting a starting protein to a high-hear process may be used to provide a modified protein or peptide with a reduced average particle size relative to the starting protein. In particular, the average particle size of the modified protein or peptide may be about 70% or less of the average particle size of the starting protein, for example about 65% or less of the average particle size of the starting protein. In some embodiments, the average particle size of the modified protein or peptide may be about 60% or less of the average particle size of the starting protein, for example about 55% or less of the average particle size of the starting protein. A decrease in average particle size of a protein may be readily determined by any suitable method which will be readily available to a person skilled in the art. One particular method which may be employed to determine the average particle size of the starting protein and the modified protein or peptide, and thus determine whether a decrease has occurred, is the use of Dynamic Light Scattering principle, for example by use of a Malvern Zetasizer (Malvern Panalytical).

The scope of the present disclosure should not be limited by reference to any specific proteins. Any suitable starting and modified proteins or peptides may be used in the compositions and methods of the present disclosure. The protein may be in the form of a protein fraction obtained, for example, from a natural source, such as a cellular or tissue source. The cellular or tissue source may be obtained from any suitable source, such as an animal or plant source. In an exemplary embodiment, the protein is whey protein isolate; the starting protein is unmodified whey protein isolate and the modified protein is a modified whey protein isolate. In another exemplary embodiment, the protein is a whey protein concentrate; the starting protein is unmodified whey protein concentrate and the modified protein is a modified whey protein concentrate. In other embodiments, the protein may be derived from a plant source, and may comprise, for example, pea-or soy-derived protein or, pea protein isolate or soy protein isolate.

Proteins of any suitable molecular weight may be employed in accordance with the present disclosure. For example, the starting protein and/or the modified protein or peptide may have a molecular weight in the range of about 500 Da to about 150 kDa. For example, the protein may have a molecular weight of up to about 500 Da, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 105 kDa, 110 kDa, 115 kDa, 120 kDa, 125 kDa, 130 kDa, 135 kDa, 140 kDa, 145 kDa, or up to about 150 kDa.

Proteins of any suitable molecular size may be employed in accordance with the present disclosure. For example, the starting protein and/or the modified protein or peptide may have a particle radius in the range of about 0.5 nm to about 5 nm. For example, the protein may have a particle radius of about 0.5 nm, 0.6 nm, 0.7 nm, 0.8 nm, 0.9 nm, 1.0 nm, 1.1 nm, 1.2 nm, 1.3 nm, 1.4 nm, 1.5 nm, 1.6 nm, 1.7 nm, 1.8 nm, 1.9 nm, 2.0 nm, 2.1 nm, 2.2 nm, 2.3 nm, 2.4 nm, 2.5 nm, 2.6 nm, 2.7 nm, 2.8 nm, 2.9 nm, 3.0 nm, 3.1 nm, 3.2 nm, 3.3 nm, 3.4 nm, 3.5 nm, 3.6 nm, 3.7 nm, 3.8 nm, 3.9 nm, 4.0 nm, 4.1 nm, 4.2 nm, 4.3 nm, 4.4 nm, 4.5 nm, 4.6 nm, 4.7 nm, 4.8 nm, 4.9 nm, or about 5.0 nm.

The modified protein(s) and/or peptide(s) may be introduced into the emulsion or composition at any stage in the preparation of the emulsion or composition such that a homogenous aqueous dispersion or slurry is formed. Those skilled in the art will be able to optimise the amount and molecular weights of the protein(s) and/or peptide(s) to be introduced without undue burden or experimentation. In some preferred embodiments, the molecular weight of the protein(s) and/or peptide(s) may be sufficiently low to facilitate microencapsulation while the amount of said protein(s) and/or peptide(s) may be sufficient to provide effective protection as the encapsulant. In the case of oil-in-water emulsions, the viscosity may also be controlled. If the viscosity is too high spray drying may be hindered. Determining the appropriate protein content and the appropriate viscosity is well within the capabilities of the skilled person.

In exemplary embodiments, the modified protein and/or peptide may be present at between about 3% (w/w) and about 30% (w/w) based on the total weight of the composition or between about 3% (w/w) and about 25% (w/w) based on the total weight of the composition. In the case of an oil-in-water emulsion, this means between about 3% (w/w) and about 30% (w/w) or between about 3% (w/w) and about 25% (w/w) based on the total weight of the aqueous phase plus the oil phase. For example, the protein and/or peptide may be present at about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% w/w based on the total weight of the composition.

In particular embodiments described herein the encapsulant comprises compounds, substances or moieties in addition to the modified protein and/or peptide. For example, the encapsulant may comprise a combination of a modified protein with one or more polysaccharide or carbohydrate components. For example, a carbohydrate with a reducing sugar functional group may be reacted with the protein. dextrose (including dextrose monohydrate), glucose, lactose, sucrose, oligosaccharide and dried glucose syrup. In a further embodiment a polysaccharide, high-methoxy pectin or carrageenan, may be added to protein-carbohydrate mixtures in some formulations. Care needs to be taken in reacting the protein and carbohydrate to ensure that the conditions do not result in extensive gelling or coagulation of the protein, as this will render the protein incapable of forming a good film.

In an exemplary embodiment, compositions of the present disclosure may be prepared by solubilising the polysaccharide or carbohydrate components of the encapsulant in an aqueous phase containing the modified protein, optionally using a high shear mixer. The mixture may then be heated to a temperature of about 50° C. to 80° C. after which time one or more antioxidants may be added if desired. The hydrophobic material may be dosed in-line to the aqueous mixture which is passed through a high shear mixer to form a coarse emulsion. The coarse emulsion may then be passed through homogenisation. If it is desired to prepare a powdered product the emulsion may be pressurised and spray-dried at an inlet temperature of about 180° C. and an outlet temperature of 80° C.

By way of example, a suitable polysaccharide and carbohydrate component may comprise maltodextrin, dextrose (including dextrose monohydrate), glucose, lactose, sucrose, oligosaccharide and dried glucose syrup, or combinations of one or more thereof. In a further embodiment a polysaccharide, high-methoxyl pectin or carrageenan, may be added to protein-carbohydrate mixtures in some formulations. Care needs to be taken in reacting the protein and carbohydrate to ensure that the conditions do not result in extensive gelling or coagulation of the protein, as this will render the protein incapable of forming a good film. The ratio (by weight) of the modified protein to the polysaccharide or carbohydrate component of the encapsulant may be, for example, about 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10.

In particular embodiments, the ratio (by weight) of the protein component of the encapsulant to the carbohydrate component of the encapsulant may be from about 1:10 to about 1:1.5. For example, the ratio of protein component to carbohydrate component may be about 1:10, 1:9.5, 1:9, 1:8.5, 1:8, 1:7.5, 1:7, 1:6.5, 1:6, 1:5.5, 1:5, 1:4.5, 1:4, 1:3.5, 1:3, 1:2.5, 1:2 or 1:1.5. The ratio of protein component to carbohydrate component may be from about 1:5 to about 1:1.5, for example about 1:4, 1:3, 1:2.9, 1:2.8, 1:2.7, 1:2.6, 1:2.5, 1:2.4, 1:2.3, 1:2.2, 1:2.1, 1:2, 1:1.9, 1:1.8, 1:1.7, 1:1.6 or 1:1.5. The ratio of protein component to carbohydrate component may be from about 1:2 to about 1:1.9, for example about 1:2, 1:1.99, 1:1.98, 1:1.97, 1:1.96, 1:1.95, 1:1.94, 1:1.93, 1:1.92, 1:1.91 or 1:1.9. In an exemplary embodiment, the ratio of protein component to carbohydrate component is about 1:1.99.

The polysaccharide or carbohydrate component may have a DE value of between about 0 and 100, about 10 and 70, about 20 and 60, or about 20 and 40. The DE value may be about 0, 5, 10, 15, 20, 25, 30, 35, 40, 45, 50, 55, 60, 65, 70, 75, 80, 85, 90, 95 or 100.

The skilled addressee will appreciate that alternative carbohydrate sources may also be employed in the encapsulant in combination with the one or more modified proteins. For example, the carbohydrate source may comprise octenylsuccinic anhydride-modified starch and one or more, or two or more, sources of reducing sugars, with dextrose equivalent values of between about 0 and 80 as has been described previously in WO2012/106777, the disclosure of which is incorporated herein by reference. Briefly, the starch may comprise primary and/or secondary modifications and may be an ester or half ester. Suitable octenylsuccinic anhydride-modified starches include, for example, those based on waxy maize and sold under the trade names PURITY GUM®, CAPSUL® IMF and HI CAP® IMF by Ingredion ANZ Pty Ltd, Seven Hills, NSW, Australia. The octenylsuccinic anhydride-modified starch may be present in an amount of less than about 18%, 17%, 16%, 15%, 14%, 13%, 12%, 11%, 10%, 9%, 8%, 7%, 6.5%, 6%, 5.5%, 5%, 4.5%, 4%, 3.5%, 3%, 2.5%, 2% or less than 1%, of the total weight of the composition.

Sources of reducing sugars are well known to those skilled in the art and include monosaccharides and disaccharides, for example glucose, fructose, maltose, galactose, glyceraldehyde and lactose. Suitable sources of reducing sugars also include oligosaccharides, for example glucose polymers, such as dextrin and maltodextrin and glucose syrup solids. The reducing sugars may also be derived from glucose syrup which typically contains not less than 20% by weight of reducing sugars.

The surface free fat content of a microencapsulated composition according to the present disclosure may be less than or about 10%, less than or about 9%, less than or about 8%, less than or about 7%, less than or about 6%, less than or about 5%, less than or about 4%, less than or about 3%, less than or about 2.5%, less than or about 2.4%, less than or about 2.3%, less than or about 2.2%, less than or about 2.1%, less than or about 2%, less than or about 1.9%, less than or about 1.8%, less than or about 1.7%, less than or about 1.6%, less than or about 1.5%, less than or about 1.4%, less than or about 1.3%, less than or about 1.2%, less than or about 1.1%, less than or about 1%, or less than about 0.8%. In particularly preferred embodiments, the surface free fat content is less than about 1.8%, for example less than about 1.5%, for example less than about 1.4%, for example less than about 1%. In some embodiments, for example wherein the protein is subject to a high shear process at an alkaline pH, for example at a pH of about 8, the surface fat free content may be less than about 1%, for example less than about 0.8%. In particular embodiments, this surface free fat content is determined in a powder derived or produced from an emulsion.

The oxidative stability of the microencapsulated compositions according to the present disclosure may be measured, for example, in terms of an induction period, for example as measured using an ML Oxipres (Mikrolab Aarhus), as described in Example 7 below (“Oxipres” is an indirect measure of potential oxidative stability). In particularly preferred embodiments, the induction period of the microencapsulated compositions according to the present disclosure when measured at 70° C. at a pressure of 5 bar is at least about 50 hours, for example at least about 60 hours, for example at least about 70 hours, for example at least about 80 hours, for example at least about 90 hours. In some embodiments, the induction period is at least about 100 hours. In some embodiments, the induction period is at least about 120 hours, for example at least about 130 hours.

Compositions and emulsions of the present disclosure comprise one or more hydrophobic materials. The term “hydrophobic material” includes pure hydrophobic compounds, hydrophobic mixtures and hydrophobic compositions. The hydrophobic material may be any hydrophobic compound or composition which it is desirable to microencapsulate in accordance with the present disclosure. Examples of hydrophobic materials which may be used in accordance with the present disclosure include bioactives such as LCPUFAs and oils comprising LCPUFAs, carotenoids, water-insoluble vitamins such as vitamins A, D, E and K, phenolic compounds, flavours and aroma compounds and edible oils. The hydrophobic material may provide one or more health benefits when administered to a subject. In particular embodiments, the hydrophobic material may be one or more LCPUFAs, or an oil(s) comprising the one or more LCPUFAs. Such oil(s) may be naturally occurring or naturally derived, or may be synthetic from genetically modified or non-genetically modified source. In the context of the present disclosure the terms “naturally occurring” and “naturally derived” include oils and lipid compositions that may be extracted from a natural source such as the organisms listed herein, or that may be derived from or modified from an oil or one or more lipids found in such natural sources. The skilled person will appreciate that scope of the present disclosure is not limited by reference to the identity or source of the hydrophobic material or the one or more LCPUFAs or oil(s) comprising the one or more LCPUFAs.

Exemplary oils that are, or can be modified to be LCPUFA-containing or LCPUFA-rich, or may be used without modification to their LCPUFA content, include oils from marine organisms such as, for example, crustaceans such as krill, molluscs such as oysters, and fish such as tuna, salmon, trout, sardines, mackerel, sea bass, menhaden, herring, pilchards, kipper, eel or whitebait. The oil may be from the roe of one or more marine organisms such as those listed herein. In exemplary embodiments, the oil is or comprises tuna oil, krill oil or a lipid extract from fish roe. In a particular embodiment, the hydrophobic material is tuna oil.

Other exemplary oils that are, or may be modified to be LCPUFA-containing or LCPUFA-rich, or may be used without modification to their LCPUFA content, include plant sources and microbial sources. Plant sources include, but are not limited to, flaxseed, walnuts, sunflower seeds, canola, safflower, soy, wheat germ, corn and leafy green plants such as kale, spinach and parsley. Microbial sources include algae and fungi.

The hydrophobic material may be present in an amount between about 0.1% and 80% of the total weight of the composition, or in an amount between about 1% and 80%, or in an amount between about 1% and 75%, or in an amount between about 5% and 80%, or in an amount between about 5% and 75%, or in an amount between about 5% and 70% of the total weight of the composition. In exemplary embodiments, where the oil is tuna oil, the oil may be present in an amount of about 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 49%, 50%, 52%, 54%, 56%, 58%, 60%, 62%, 64%, 66%, 68%, 70%, 72%, 74%, 76%, 78% or 80% of the total weight of the composition.

Hydrophobic materials comprising LCPUFAs typically comprise one or more omega-3 fatty acids and/or one or more omega-6 fatty acids, or mixtures thereof. The fatty acids may include DHA, AA, EPA, DPA and/or stearidonic acid (SDA), or mixtures thereof. In one embodiment, the fatty acids comprise DHA and EPA.

Compositions contemplated by the present disclosure may further comprise additional components, for example, antioxidants, anti-caking agents, flavouring agents, colouring agents, vitamins, minerals, amino acids, chelating agents and the like.

Suitable antioxidants are well known to those skilled in the art, and may be water soluble or oil soluble. Suitable water soluble antioxidants include, for example, sodium ascorbate, calcium ascorbate, potassium ascorbate, ascorbic acid, glutathione, lipoic acid and uric acid. In an embodiment the water soluble antioxidant may be present in the composition in a range of about 0-10% wt/wt of the total composition. Suitable oil soluble antioxidants include, for example, tocopherols, ascorbyl palmitate, tocotrienols, phenols, polyphenols and the like. In an embodiment the oil soluble antioxidant is present in the oil phase in a range of about 0-10% wt/wt of the total composition.

Anti-caking agents that are compatible with the compositions of the present disclosure will be well known amongst those skilled in the art and include calcium phosphates, such as tricalcium phosphate and carbonates, such as calcium and magnesium carbonate and silicon dioxide

The compositions may further comprise one or more low molecular weight emulsifiers. Suitable low molecular weight emulsifiers include, for example, mono- and di-glycerides, lecithin and sorbitan esters. Other suitable low molecular weight emulsifiers will be well known to those skilled in the art. The low molecular weight emulsifier may be present in an amount between about 0.1% and 3% of the total weight of the composition, or in an amount between about 0.1% and about 2%, or in an amount between about 0.1% and 0.5%, or in an amount between about 0.1% and 0.3%, of the total weight of the composition. For example, the low molecular weight emulsifier may be present in an amount of about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2% of the total weight of the composition.

Compositions contemplated herein may be formulated for administration to subjects by any suitable route, typically oral administration. The composition may be in liquid or solid form, and may be consumed as such (for example in the form of a syrup or other suitable liquid, or as capsules or other suitable solid form). Alternatively, the compositions may be incorporated into food or beverage products.

It will be appreciated by persons skilled in the art that numerous variations and/or modifications may be made to the invention without departing from the spirit or scope of the invention as broadly described. The present embodiments are, therefore, to be considered in all respects as illustrative and not restrictive.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be further described in greater detail by reference to the following specific examples, which should not be construed as in any way limiting the scope of the invention.

### EXAMPLES

### Example 1—Modification of Whey Protein Isolate

Whey protein isolate (WPI) and whey protein concentrate (WPC) were each dissolved in water at 10% (w/w). The pH of some of the WPI solution was adjusted to 8. The solutions (WPI, WPC and WPI (pH 8)) were agitated under gentle shear at 50° C. for 30 minutes. Subsequently, the mixtures were homogenised at 1500 bar (150 mPa) for 6 passes to induce modification of the whey protein. Ice packs were used to maintain the temperature of the WPI and WPC below 60° C. during homogenisation.

### Example 2—Particle Size Analysis of Aqueous Protein Solutions

Protein particle sizes of 1.0% w/w solutions of unmodified whey protein isolate (uWPI) and modified whey protein isolate (mWPI) and unmodified whey protein concentrate (uWPC) and modified whey protein concentrate (mWPC) obtained in Example 1 were measured using a Malvern Zetasizer via Dynamic Light Scattering principles. Backscatter (BS) examines a wide spectrum of particle sizes whilst Forward scatter (FS) captures larger particle size ranges. Results are shown in Table 1 below.

TABLE 1

| Z-Av | | mWPI | mWPI | | mWPC |
|---|---|---|---|---|---|
| (nm) | uWPI | (native pH) | (pH 8) | uWPC | (native pH) |
| BS | 88.4 ± 4.3 | 45.3 ± 6.6 | 44.5 ± 14.7 | 84.7 ± 0.9 | 52.0 ± 0.3 |
| FS | 739.2 ± 31.2 | 367.8 ± 52.4 | 293.3 ± 147.4 | 258.2 ± 9.8 | 150.2 ± 6.3 |

All samples are polydisperse, therefore presence of very large particles can considerably influence the average particle size (Z-Av) as illustrated in Table 1. With modification of Whey Protein Isolate (mWPI), there is a significant reduction in protein particle size in contrast to the unmodified version (uWPI), regardless of pH. This is thought to be due to pressure modification largely separating soluble protein aggregates. uWPC has similar average particle size to uWPI but substantially smaller soluble protein aggregates as seen shown in the FS result. The extent of pressure modification on particle size reduction is similar when exerted on Whey Protein Concentrate (WPC). There is an almost 2-fold reduction in average protein particle size with 1500 bar/6 passes pressure modification for both WPI and WPC.

### Example 3—Surface Charge Analysis of Aqueous Protein Solutions

Surface charge of 1.0% w/w aqueous solutions/dispersions of uWPI and mWPI obtained in Example 1 at both native pH (i.e. without any pH adjustment) and pH 8 was measured using a Zetasizer, which measures electrokinetic potential. Results are shown in Table 2 below.

TABLE 2

| | | mWPI | mWPI |
|---|---|---|---|
| ZP (mV) | uWPI | (native pH) | (pH 8) |
| 1.0% w/w | −17.0 ± 0.6 | −16.34 ± 1.1 | −18.8 ± 0.9 |

The slight difference in surface charge (Zetapotential; ZP) of mWPI solutions can be attributed to the reduction in soluble protein aggregates. An alkaline pH of 8 was used to improve the hydration properties of the WPI and a more negative surface charge was observed.

### Example 4—Interfacial Tension of Aqueous Protein Solutions With Corn Oil

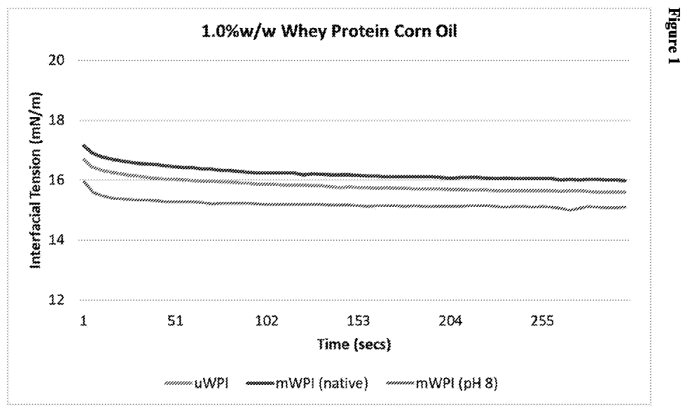

Interfacial tension of 1.0% w/w aqueous solutions of uWPI and mWPI obtained in Example 1 are shown in FIG. 1.

The interfacial tension of corn oil with water is approximately 27 mN/m. 1.0% w/w WPI solutions effectively reduced the interfacial tension of com oil with water, demonstrating good surface activity & adsorption behaviour at the interface. mWPI solutions with lower average particle size, better hydration (pH 8) and therefore, faster diffusion to the interface, further reduced interfacial tension values as opposed to uWPI solution.

### Example 5—Protein/Oil Emulsions

1% w/w aqueous solutions of uWPI and mWPI as obtained in Example 1 and refined tuna oil containing mixed natural tocopherols were used to prepare oil-in water emulsions (protein to oil weight ratio of 1:3). A mixture of WPI solution and tuna oil was coarsely homogenised using a UltraTurrax at 10,000 RPM for 10 minutes. Median oil globule size d(0.5) and average oil globule size D[4.3] (in micrometers) were measured using a Particle Sizer (Malvern Instruments, Mastersizer MS3000) based on laser diffraction principles; the results are shown in Table 3 below. “EAI” as shown in Table 3 refers to the emulsion activity index and “ESI” refers to the emulsion stability index. EAI reflects the ability of the protein to adsorb at the oil-water interface and ESI in resisting instability of emulsion to, for example, flocculation and creaming.

TABLE 3

| | | | mWPI | |
|---|---|---|---|---|
| | | uWPI | (native) | mWPI (pH 8) |
| Day 0 | d(0.5) | 4.5 ± 0.5 | 4.0 ± 0.7 | 2.5 ± 0.3 |
| | D[4, 3] | 6.3 ± 2.1 | 5.1 ± 1.2 | 4.1 ± 0.4 |
| Day 4 | d(0.5) | 6.2 ± 0.6 | 5.1 ± 1.1 | 3.1 ± 0.3 |
| | D[4, 3] | 16.5 ± 3.7 | 7.2 ± 0.9 | 6.5 ± 1.5 |
| Day 7 | d(0.5) | 9.2 ± 1.0 | 11.0 ± 3.6 | 3.9 ± 1.0 |
| | D[4, 3] | 28.0 ± 11.9 | 27.7 ± 19.0 | 16.5 ± 9.0 |
| EAI | | 0.0099 ± | 0.0104 ± | 0.0114 ± |
| (m²/g) | | 0.0004 | 0.0015 | 0.0007 |
| ESI | | 38.78 ± | 85.37 ± | 362.9 ± |
| (mins) | | 3.58 | 26.19 | 141.62 |

The median oil globule size d(0.5) and average oil globule size D[4.3] were smaller with mWPI (pH 8). Both parameters increased over time regardless of modification or pH treatment, but the magnitude of change was least with mWPI (pH 8) after 1-week storage duration. This corresponded to a better interfacial activity of mWPI (pH 8) solution as illustrated in Example 4 and adsorption behaviour that conferred good emulsion stability over time.

### Example 6—Encapsulation of Hydrophobic Material Using Unmodified and Modified Protein Encapsulants

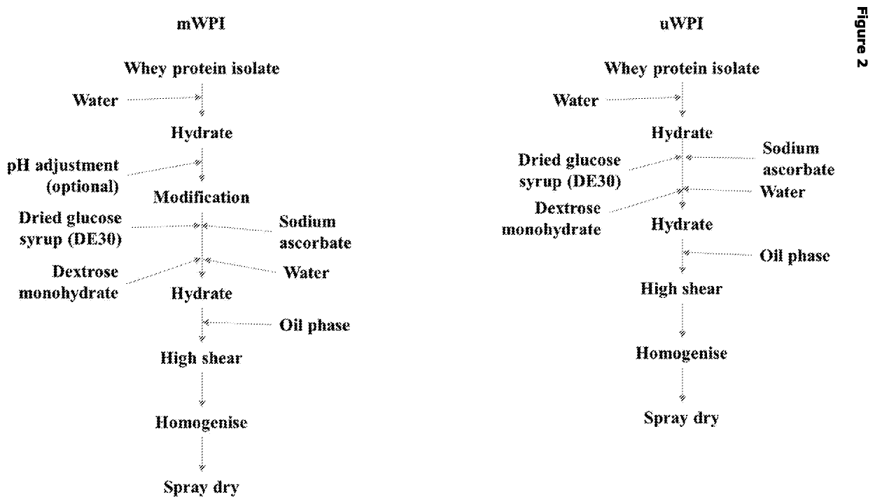

Unmodified whey protein isolate (uWPI) and modified whey protein isolate (mWPI) obtained in Example 1 were used to prepare microencapsulated compositions. A refined tuna oil containing mixed natural tocopherols was used as the hydrophobic core material. The formulations of each of the microencapsulated compositions are shown in Table 4 below. The preparation methods of each composition are depicted in FIG. 2, and discussed in detail below.

TABLE 4
Formulation of tuna oil microencapsulated powders

| | Whey Protein- |
|---|---|
| | microencapsulated powder |
| WPI | 15.00 |
| Dextrose monohydrate | 14.50 |
| Dried glucose syrup (DE | 15.10 |
| value of 30) | |
| Sodium ascorbate | 5.35 |
| Tuna oil | 50.00 |
| Antioxidant | 0.05 |
| Total | 100% |

Preparation of Microencapsulated Powdered Composition Using uWPI and Carbohydrate Encapsulant (“uWPI-Microencapsulated Powder”)

WPI (15.00% (w/w)), dextrose monohydrate (14.50% (w/w)), dried glucose syrup (with DE value 30) (15.10% (w/w)) and sodium ascorbate (5.35% (w/w)) were dissolved in water. This aqueous phase was agitated under gentle shear at 50° C. for 35 minutes. Tuna oil containing antioxidants (50.05% (w/w)) was then added, following which an emulsion was prepared as follows: a coarse emulsion was produced using high shear mixing at 10,000 rpm for 10-15 minutes, followed by two-stage homogenisation at 400/200 bar (600 bar total) for 3 passes to produce a fine emulsion. The final oil-in-water emulsion was spray dried using a benchtop spray drier with inlet and outlet temperature(s) of 170 and 90-100° C., respectively. The produced powder was packed in an aluminium sachet under N₂ as a protective gas. The uWPI powder was stored at 25° C. before use. The total oil loading in the uWPI powder was 50% (w/w).

Preparation of Microencapsulated Powdered Composition Using mWPI and Carbohydrate Encapsulant (“mWPI-Microencapsulated Powder”)

WPI was modified as described in Example 1. To the aqueous phase of mWPI dextrose monohydrate (14.50% (w/w)), dried glucose syrup (with DE value 30) (15.10% (w/w)) and sodium ascorbate (5.35% (w/w) were added at 50° C. Refined tuna oil containing mixed natural tocopherols was added (50.05% (w/w)), following which an emulsion was prepared as follows: a coarse emulsion was produced using high shear mixing at 10,000 rpm for 10-15 minutes, followed by two-stage homogenisation at 400/200 bar (40/20 mPa) (600 bar total (60 mPa)) for 3 passes to produce a fine emulsion. The final oil-in-water emulsion was spray dried using a benchtop spray drier with inlet and outlet temperature ranges of 170 and 90-100° C., respectively. The produced powder was packed in an aluminium sachet under N₂ as a protective gas. The total oil loading in the mWPI powder was 50% (w/w).

Preparation of Microencapsulated Powdered Composition Using mWPI (pH 8 Solution) and Carbohydrate Encapsulant (“mWPI-Microencapsulated Powder (pH 8)”).

WPI solution was adjusted to pH 8 and modified as described in Example 1. To the aqueous phase of mWPI dextrose monohydrate (14.50% (w/w)), dried glucose syrup (with DE value 30) (15.10% (w/w)) and sodium ascorbate (5.35% (w/w) were added at 50° C. Refined tuna oil containing mixed natural tocopherols was added (50.05% (w/w)), following which an emulsion was prepared as follows: a coarse emulsion was produced using high shear mixing at 10,000 rpm for 10-15 minutes, followed by two-stage homogenisation at 400/200 bar (40/20 mPa) (600 bar total (60 mPa)) for 3 passes to produce a fine emulsion. The final oil-in-water emulsion was spray dried using a benchtop spray drier with inlet and outlet temperature ranges of 170 and 90-100° C., respectively. The produced powder was packed in an aluminium sachet under N₂ as a protective gas. The total oil loading in the mWPI powder was 50% (w/w).

### Example 7—Assessment of Surface Free Fat and Oxidative Stability of Tuna Oil in Microcapsule Powders

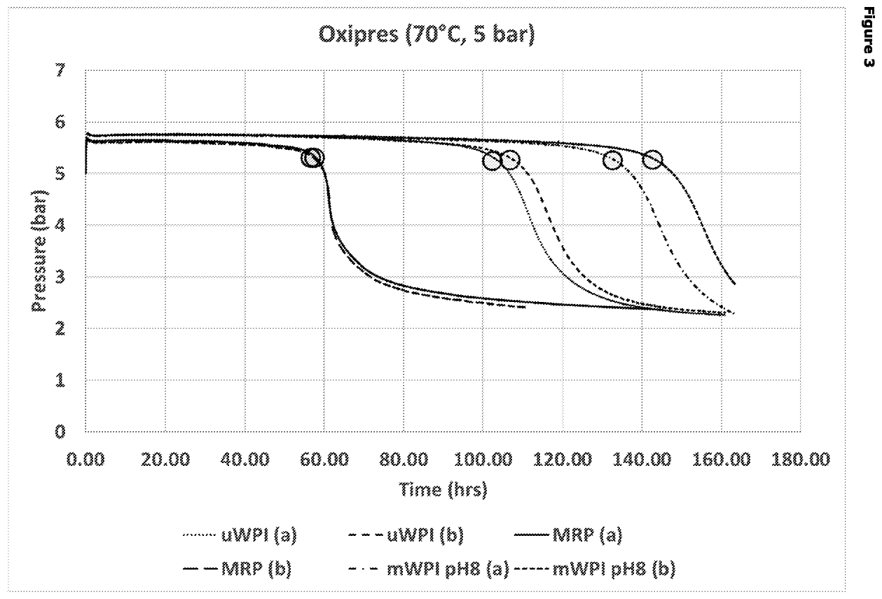

The oxidative stability of the microencapsulated powders were analysed using Oxipres as a fast and reliable instrumental method. The microencapsulated powder with a total 4 g of oil was sealed in a vessel and heated at 70° C. under oxygen at 5 bars (0.5 mPa). The time when the oxygen pressure in the vessel started to decline was recorded as induction period (IP), suggesting the occurrence of oxidation. FIG. 3 shows Oxipres analysis of the omega-3 containing microencapsulated powders compared to omega-3 oil-containing microcapsule powders encapsulated with Maillard reaction products (MRPs), as described in U.S. Pat. No. 7,374,788B2.

Percentage surface free fat was measured by subjecting the powder to petroleum spirit for a brief period (15 minutes) to extract surface free fat; the wall material/encapsulated oil was removed via filter paper and the solvent containing the “washed” fat was then evaporated and the residual weight i.e. the oil was divided by the weight (in g) of powder used, multiplied by 100% to give the surface free fat %. The results are shown in Table 5 below.

TABLE 5

| | | mWPI | mWPI |
|---|---|---|---|
| | uWPI | (native pH) | (pH 8) |
| Total fat | 49.0 | 49.0 | 49.0 |
| content (%) | | | |
| Surface Free | 2.2 ± 0.7 | 1.3 ± 0.5 | 0.6 ± 0.1 |
| Fat (%) | | | |

As shown in FIG. 3, the induction period was greatly increased by use of the modified protein encapsulant. As shown in Table 5, mWPI microencapsulated powders present significantly lower surface free fat (SFF) values than uWPI. mWPI (pH 8) had less than 1% SFF which meets typical powder requirements, whilst also providing a high (˜50%) oil load and good oxidative stability. All samples showed good oxidative stability via Oxipres (70° C., 5 bar) with more than 100 hours IP.

Good oxidative stability in terms of primary and secondary oxidative properties is further illustrated in Table 6 below, where microencapsulated powders were subjected to 4 weeks of exposure to 40° C. temperature. By the end of 4 weeks, all samples were well within specifications for peroxide value (POV; 5 meq O₂/Kg fat) and p-Anisidine value (p-AV; 20) values.

TABLE 6

| | | | | Accelerated | | | Accelerated | | |
|---|---|---|---|---|---|---|---|---|---|
| | Baseline | | | 2-weeks | | | 4-weeks | | |
| | | POV | | | POV | | | POV | |
| Sample | | (meq | | | (meq | | | (meq | |
| (50% | Total | O₂/ | | Total | O₂/ | | Total | O₂/ | |
| oil | Fat | Kg | | Fat | Kg | | Fat | Kg | |
| load) | (%) | fat) | PAV | (%) | fat) | PAV | (%) | fat) | PAV |
| uWPI | 49.2 | 0.8 | 4.8 | 49.2 | 0.9 | 3.9 | 49.4 | 0.7 | 2.8 |
| mWPI | 49.5 | 0.6 | 4.3 | 49.8 | 0.5 | 4.1 | 49.3 | 0.6 | 3.1 |
| (native) | | | | | | | | | |
| mWPI | 49.7 | 1.2 | 4.5 | 49.5 | 0.5 | 3.3 | 49.7 | 0.8 | 2.4 |
| (pH 8) | | | | | | | | | |

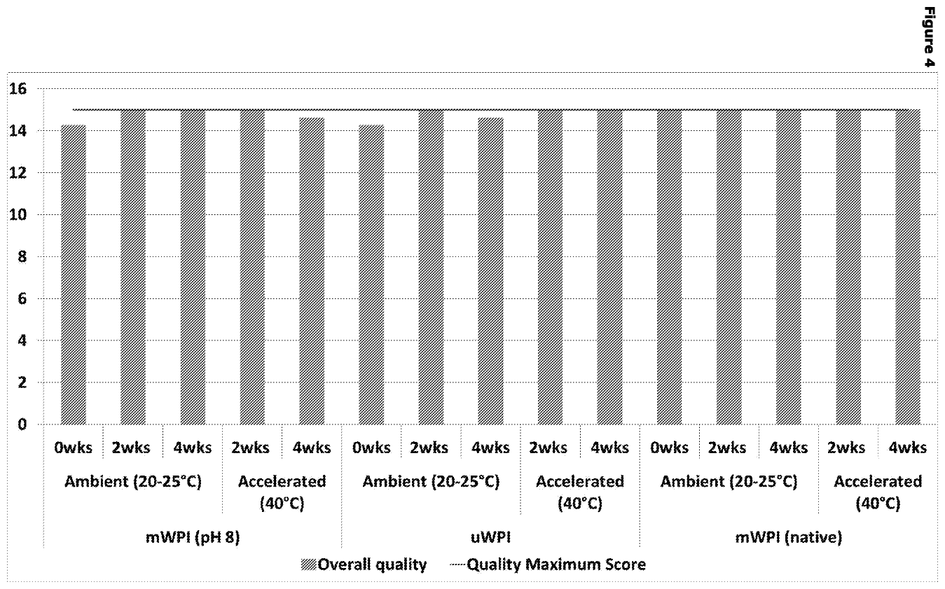

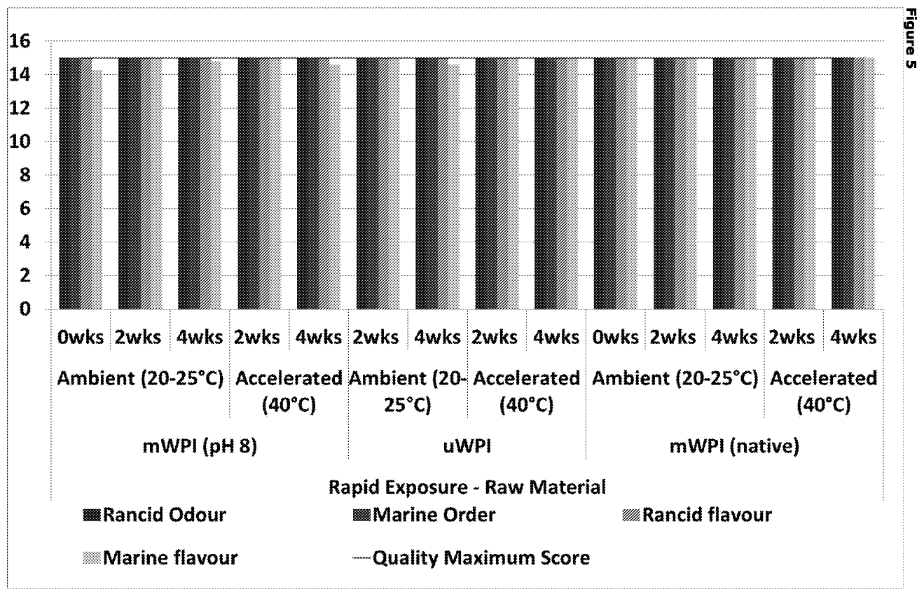

Sensory attributes of the microencapsulated uWPI & mWPI were also assessed over a 4 week rapid exposure period. Results are shown in FIGS. 4 and 5. A maximum score of 15 indicates excellent quality/attribute. Overall quality (FIG. 4) was rated as good across all samples by end of the storage period with no perceivable rancid and marine odours and flavours (FIG. 5).

1. A microencapsulated composition comprising one or more hydrophobic materials encapsulated by an encapsulant, wherein the encapsulant comprises one or more modified proteins and/or peptides; and wherein the one or more modified proteins and/or peptides are obtained from a starting protein by subjecting the starting protein to a high shear process, such that the average particle size of the one or more modified proteins and/or peptides is reduced relative to the starting protein.

2. The microencapsulated composition according to claim 1, wherein the average particle size of the one or more modified proteins and/or peptides is about 70% or less of the average particle size of the starting protein, or about 65% or less of the average particle size of the starting protein.

3. (canceled)

4. The microencapsulated composition according to claim 1, having a surface fat free content of less than about 1.8%, less than about 1%, or less than about 0.8%.

5-7. (canceled)

8. The microencapsulated composition according to claim 1, wherein the high shear process: is carried out at a pH of about 8; comprises subjecting the starting protein to a pressure of from about 20 mPa to about 300 mPa; is a homogenisation process; and/or is a microfluidisation process.

9.-11. (canceled)

12. The microencapsulated composition according to claim 1, wherein the one or more modified proteins and/or peptides are in the form of a protein fraction.

13. The microencapsulated composition according to claim 1, wherein the modified protein is modified whey protein.

14. The microencapsulated composition according to claim 1, wherein the encapsulant further comprises one or more carbohydrates, optionally selected from glucose syrup and dextrose monohydrate, or a combination thereof.

15. (canceled)

16. The microencapsulated composition according to claim 1, wherein the one or more modified proteins and/or peptides are present at from about 3% w/w to about 25% w/w based on the total weight of the composition.

17. The microencapsulated composition according to claim 14 wherein the ratio of the modified protein component of the encapsulant to the carbohydrate component of the encapsulant is in the range of about 1:10 to 1:1.

18. (canceled)

19. The microencapsulated composition according to claim 1, wherein the hydrophobic material comprises one or more long-chain polyunsaturated fatty acids (LCPUFAs).

20. (canceled)

21. The microencapsulated composition according to claim 19, wherein the LCPUFAs are present in triglyceride form and/or in one or more LCPUFA-containing oils.

22.-25. (canceled)

26. The microencapsulated composition according to claim 1, wherein the composition is in the form of an oil-in-water emulsion or a spray dried powder.

27. (canceled)

28. A method for protecting a hydrophobic material from oxidative degradation, for improving the oxidative stability of a hydrophobic material and/or for reducing the surface free fat of a microencapsulated composition comprising a hydrophobic material, comprising:
subjecting a starting protein to a high shear process producing one or more modified proteins and/or peptides, such that the average particle size of the one or more modified proteins and/or peptides is reduced relative to the starting protein; and
encapsulating the one or more hydrophobic materials with an encapsulant comprising the one or more modified proteins and/or peptides.

29.-30. (canceled)

31. The method according to claim 28, wherein the average particle size of the one or more modified proteins and/or peptides is about 70% or less of the average particle size of the starting protein or about 65% or less of the average particle size of the starting protein.

32.-33. (canceled)

34. The method according to claim 28, wherein the high shear process is carried out at a pH of about 8.

35. The method according to claim 28, wherein the hydrophobic material comprises one or more LCPUFAs.

36. (canceled)

37. A stable emulsion comprising a hydrophobic material, wherein said emulsion further comprises one or more modified proteins and/or peptides, and wherein the one or more modified proteins and/or peptides are obtained from a starting protein by subjecting the starting protein to a high shear process, such that the average particle size of the one or more modified proteins and/or peptides is reduced relative to the starting protein.

38. The stable emulsion according to claim 37, wherein the average particle size of the one or more modified proteins and/or peptides is about 70% or less of the average particle size of the starting protein, or about 65% or less of the average particle size of the starting protein.

39.-40. (canceled)

41. The stable emulsion according to claim 37, wherein the high shear process is carried out at a pH of about 8.

42. The stable emulsion according to claim 37, wherein the hydrophobic material comprises one or more LCPUFAs.

43.-44. (canceled)
